(19) United States
(12) Patent Application Publication
(10) Pub. No.: US 2005/0059728 A1
(43) Pub. Date: Mar. 17, 2005

(54) Dithiolane derivatives for immobilising biomolecules on noble metals and semiconductors

(76) Inventors: Hartmut Seliger, Elchingen (DE); Tobias Prokein, Elchingen (DE)

Correspondence Address: KLAUBER & JACKSON, 411 Hackensack Ave., 4th Floor, Hackensack, NJ, 07601, US

(21) Appl. No.: 10/926,575

(22) Filed: Aug. 26, 2004

Related U.S. Application Data
(63) Continuation of application No. PCT/EP03/01968, filed on Feb. 26, 2003.

(30) Foreign Application Priority Data
Mar. 5, 2002 (DE) 102 09 641.4
Nov. 4, 2002 (DE) 102 51 229.9

(51) Int. Cl. A61K 31/385; C07D 339/02

(52) U.S. Cl. 514/440; 549/39

(57) ABSTRACT

The present invention relates to dithiolane derivatives of formula A, 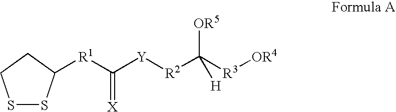 to conjugates of the dithiolane derivatives with organochemical or biochemical molecules, to processes for the preparation of the dithiolane derivatives and to the conjugates, to a coated noble metal or semiconductor structure comprising a noble-metal-coated substrate or a semiconductor carrier and a dithiolane derivative immobilised thereon or a conjugate according to the invention, to a biochip containing the noble metal or semiconductor structure according to the invention, and to the use of the dithiolane derivatives according to the invention for immobilising organochemical or biochemical molecules on noble metals, noble metal alloys or semiconductors.

The present invention relates to dithiolane derivatives, to conjugates of the dithiolane derivatives with organochemical or biochemical molecules, to processes for the preparation of the dithiolane derivatives and of the conjugates, to a coated noble metal or semiconductor structure comprising a noble-metal-coated substrate or a semiconductor carrier and a dithiolane derivative immobilised thereon or a conjugate according to the invention, to a biochip containing the noble metal or semiconductor structure according to the invention, and to the use of the dithiolane derivatives according to the invention for immobilising organochemical or biochemical molecules on noble metals, noble metal alloys or semiconductors.

The chemistry of the anchoring of biochemical molecules, especially oligonucleotides, to surfaces plays a decisive role in many analytical and biomedical applications. This is the case, for example, for the preparation of oligonucleotide libraries on carriers of flat material (“oligonucleotide chips” or “DNA microarrays” or “oligonucleotide microarrays”), which have in the meantime become indispensable tools of modern biomedical diagnostics (Niemeyer et al. (1999) Angew. Chem. 19: 3039-3043), but likewise for biomolecules which are immobilised on other types of carrier, for example particulate, especially spherical, carriers. It is immaterial whether the biomolecules, such as, for example, oligonucleotides, are composed of monomeric or oligomeric synthons at the surface or whether they are applied to the surface in the form of completed chains of the monomeric units, for example in the form of oligonucleotide chains, after synthesis has been carried out. It is important, however, that anchoring to the surface should be stable and suitable for the intended function, that is to say, for example, that it is ensured that the immobilised organochemical or biochemical molecules, such as oligonucleotides, peptides, etc., do not become detached under the conditions of use even when they are used several times or many times. On the other hand, however, it is desirable in some applications for the grafted (immobilised) molecule to be capable of being cleaved off under particular conditions, for example for the purpose of analysis.

A large number of possibilities for preparing biomolecules, especially oligonucleotides, applied to glass, plastics and other non-metallic carriers has already been described in the prior art (Wölfl (2000) BioChip-Technologien, Laborwelt, Transcript 3: 12-20). Of these, oligonucleotide chips on noble metal, preferably gold surfaces, are of particular interest, inter alia, on account of their simple handling and the possibility of producing so-called “self-assembled monolayers” (SAM), which permit efficient control of the density of the immobilised molecules, as a result of which a high signal intensity can be achieved. Such SAMs of organic molecules on noble metal surfaces are usually produced by reaction with thiols (Bain et al. (1988) J. Am. Chem. Soc. 111: 321-335), which then in turn, as anchoring groups, bring about the immobilisation of the desired molecules such as, for example, oligonucleotides (Herne et al., J. Am. Chem. Soc. 119: 8916-8920).

The bond strength of the interaction of elemental gold with (alkane)thiols is in the order of magnitude of from about 30 to about 40 kcal/mol (Nuzzo et al. (1986) J. Am. Chem. Soc. 109: 2358-2368; Nuzzo et al. (1990) J. Am. Chem. Soc. 112: 558-569). The interaction of gold and other noble metals with vicinal dithiol compounds should be substantially more stable, however, because the bond strength per molecule here increases (Wang et al. (1998) J. Phys. Chem. B, 102: 9922-9927; Cheng et al. (1995) Anal. Chem. 67: 2767-2775).

Letsinger et al. (Bioconjugate Chem. 11: 289-291, 2000) describe a vicinal dithiol compound and its interaction with gold surfaces. However, the anchoring group is a steroid derivative which is obtainable synthetically only with difficulty. When such sterically problematical groups are used, it is virtually impossible to produce SAMs on the noble metal surface, and the compounds described in this prior art have therefore not been used to produce biochips.

A number of lipoic acid constructs are known in the prior art, which constructs are used especially for immobilising biomolecules on gold surfaces; see e.g. Madoz et al. (1997) J. Am. Chem. Soc. 119: 1043-1051; Blonder et al. (1998) J. Am. Chem. Soc. 120: 9335-9341; Stevenson et al., (2002) J. Nanosci. Nanotech. 2; 397-404; Kim et al. (2002) Langmuir 18: 1460-1462. However, these lipoic acid conjugates have clear disadvantages. In some cases, the (bio)molecule to be conjugated with the lipoic acid derivative must first be correspondingly derivatised in order to render it amenable to the reaction with the lipoylating reagent. Moreover, it is not possible with the known lipoylating reagents to introduce lipoic acid moieties into any desired amine or hydroxyl groups using standard chemical processes.

Holupirek (dissertation, Ulm University, section Polymere, 1976) describes the possibility of attaching lipoic acid to oligonucleotides as a substituent and using this substituent to purify the target oligonucleotide from the product mixture of the solid-phase synthesis by means of affinity chromatography on organomercury columns. However, the process proved to be impracticable, which was due especially to the only very inefficient introduction of the lipoic acid moiety into oligonucleotides. Moreover, the regeneration of the lipoylated oligonucleotide after the affinity purification was difficult.

Accordingly, the object underlying the present invention is to provide compounds which are particularly suitable for the formation of SAMs on noble metal or semiconductor surfaces, which compounds can be introduced simply and efficiently into the organochemical or biochemical molecules that are to be immobilised.

This object is achieved by the embodiments of the present invention characterised in the claims.

There is provided according to the invention in particular a dithiolane derivative of formula A

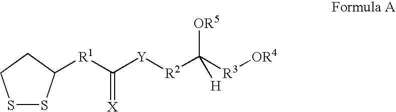

In the dithiolane derivative of the present invention, a dithiolane ring is covalently bonded via a bridging member R¹ to a carbonyl (X═O) or sulfonyl (X═S) function derived from an acid moiety. The bridging member R¹ does not have to be present, but may consist of an optionally substituted or unsubstituted aliphatic or heteroaliphatic chain which may be saturated or unsaturated, straight-chain or branched, or may be an aromatic moiety or contain aromatic moieties and will typically contain from 0 to 20 carbon atoms. Preferred bridging members are, for example, corresponding single or multiple methylene groups, such as methylene, di-, tri-, tetra-, penta- and hexa-methylene. A particularly preferred bridging member R¹ is a tetramethylene group.

The carbonyl or sulfonyl group associated with the acid moiety is bonded via an amide (Y═NH) or ester (Y═O) bond to the amino group or hydroxyl group of a diol spacer.

The moieties R² and R³ of the diol spacer may be identical or different and in principle are not subject to any particular limitations. They may be selected independently of one another from aliphatic and heteroaliphatic groups which may be saturated or unsaturated, straight-chain or branched and/or optionally substituted and usually contain from 1 to 10 carbon atoms. R² and R³ may, however, also be corresponding substituted or unsubstituted aromatic groups or may contain such groups. From the point of view of the formation of SAMs by the dithiolane derivative according to the invention or conjugates derived therefrom on corresponding surfaces, such as noble metals, R¹, R² and R³ are preferably so selected to give a number of carbon atoms from the dithiolane group to the group —OR⁴ or —OR⁵ of from about 5 to about 20, particular preference being given to corresponding saturated aliphatic or heteroaliphatic groups as groups that are not very problematical sterically. The moiety R² of the diol spacer is therefore preferably selected from (CH₂)ₙ groups, wherein n is an integer from 1 to 10, more preferably from 1 to 5. Particular preference is given to spacers in which R² is methylene (CH₂). The moiety R³ is accordingly preferably selected from (CH₂)ₘ groups, wherein m is an integer from 1 to 10, more preferably from 1 to 5. Particular preference is given to spacers where R³=methylene. A particularly preferred diol spacer is 3-aminopropane-1,2-diol or, in general, a spacer in which R² and R³ are methylene.

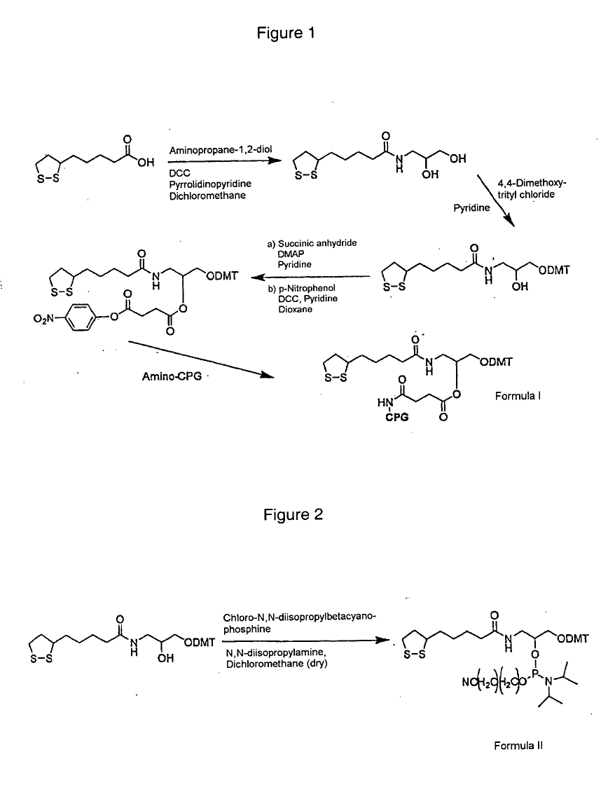

The hydroxyl groups of the diol spacer in the dithiolane derivative according to the invention of formula A above are substituted by identical or different substituents or are unsubstituted. According to the invention, therefore, the moieties R⁴ and R⁵ may, for example, both be a hydrogen atom or an acid-labile protecting group, for example dimethoxytrityl or a related group. The moieties R⁴ and R⁵ are preferably different. In a particularly preferred embodiment, which is suitable, for example, as the starting point for a further derivatisation to obtain particularly advantageous lipoylating reagents, R⁴ is H and R⁵ is an acid-labile protecting group or vice versa (see in this connection the specific examples of the reaction schemes shown in FIGS. 1 to 3). Suitable acid-labile protecting groups, such as dimethoxytrityl and similar groups, are known to a person skilled in the art and are described, for example, by Seliger in Beaucage et al. (ed.) Current Protocols in Nucleic Acid and Chemistry, Vol. 1: 2.3.1-2.3.34, Wiley & Sons, New York, 2000. The relevant content of the disclosure of this publication is incorporated in its entirety into the present invention by reference. Such protecting groups are suitable especially for coupling reactions with nucleic acids such as oligonucleotides. Further substituents of the hydroxyl groups of the diol spacer are amino and/or hydroxy coupling groups. Such coupling groups are known in the art and are correspondingly activatable moieties, for example those moieties which are used for anchoring biomolecules, especially oligonucleotides, but also low molecular weight organochemical molecules, to carriers containing amino or hydroxyl groups, such as, for example, corresponding polymer carriers, for example long-chain amino-CPG, especially aminopropyl-CPG. Such coupling groups are especially dicarbonyl groups such as, for example, a succinyl group. Other amino and/or hydroxy coupling groups which are preferably used according to the present invention are phosphoramidite groups, for example a beta-cyanoethoxy-diisopropylaminophosphane moiety. Accordingly, the coupling group, as an activatable moiety, enables the dithiolane-bridging member-spacer unit to be attached to a hydroxyl or amino group.

Particularly preferred dithiolane derivatives of the present invention are those in which R⁴ is H or an acid-labile protecting group, for example a dimethoxytrityl group, and R⁵ is a dicarbonyl group, especially a succinyl group. It is, however, also possible, conversely, for R⁵ to be H or an acid-labile protecting group and R⁴ to be a dicarbonyl group. In particularly preferred embodiments of such a dithiolane derivative, the dicarbonyl group, for example a succinyl group, is bonded to a polymer carrier via an amide or ester bond. Such a particularly preferred dithiolane derivative of the present invention is a dithiolane modifier shown in the following formula I, in this case a lipoic acid modifier:

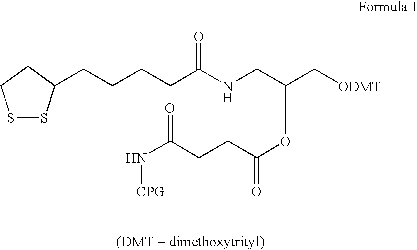

In another specific embodiment of the dithiolane derivative of the present invention, R⁴ in formula A above is H or an acid-labile protecting group (e.g. a dimethoxytrityl group) and R⁵ in formula A above is a phosphoramidite group, it also being possible for the substituents R⁴ and R⁵ to have the opposite meanings. A N,N-diisopropyl-beta-cyanoethoxyphosphoramidite as R⁵ or R⁴ is very particularly preferred.

A specific embodiment of the dithiolane derivative is a lipoic acid-substituted phosphoramidite reagent of the following formula:

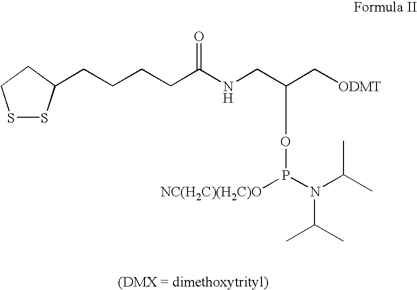

The dithiolane derivative according to the invention is excellently suitable for the introduction of dithiolane groups (with an attached bridging member) into (low molecular weight) organochemical compounds and biochemical molecules which contain amino and/or hydroxy groups. The present invention accordingly further provides a conjugate of the dithiolane derivative according to the above definition and an organochemical or biochemical molecule containing at least one amino or hydroxyl group, which molecule accordingly replaces the group —R⁴ or the group —R⁵ in formula A above via the amino or hydroxyl group.

The corresponding lipoic acid derivative is very particularly suitable for this application according to the invention of the dithiolane derivative, because this substituent, when applied to a noble metal surface, such as a gold surface, provides a high bond strength. Moreover, derivatives of the present invention derived from lipoic acid form particularly readily controllable SAMs. Moreover, lipoic acid as a starting material is readily and inexpensively obtainable. Furthermore, the lipoic acid substituent is a naturally occurring biological molecule and therefore constitutes a lower or no toxicity risk in, for example, medical-therapeutic or medical-diagnostic applications of immobilised or non-immobilised corresponding conjugates, for example oligonucleotides, especially in an antisense or antigen therapy. However, it is of course also possible to use other compounds containing the characteristic dithiolane moiety as a substitutent in the dithiolane derivative according to the invention and accordingly also in the conjugate according to the invention.

The organochemical or biochemical molecule in the conjugate according to the invention is generally understood to be any such compounds having at least one amino or hydroxyl group. According to the invention, the organochemical molecule is understood to be, for example, a low molecular weight organochemical compound having a molecular weight of, for example, <1500 Da. Biochemical molecules in the conjugate according to the invention are especially those which contain at least one component originating from a naturally occurring compound. Preferred biochemical molecules are accordingly peptides, oligopeptides, polypeptides, especially proteins, monosaccharides, oligosaccharides, polysaccharides, lipids and their constituents, especially fatty acids, as well as nucleic acids and their constituents, that is to say nucleosides, nucleotides, oligonucleotides and polynucleotides. Of course, it is also possible very generally to use mixed forms of the above-mentioned biochemical molecules, for example lipoproteins, glycoproteins etc. Particularly preferred biochemical molecules in the conjugate according to the invention are nucleic acids and their constituents, that is to say nucleosides, nucleotides, oligonucleotides and polynucleotides. These may be both deoxyribonucleotide species and ribonucleotide species and mixed forms thereof. Furthermore, the nucleic acid species according to the invention may be single-stranded or double-stranded or both single-stranded and double-stranded. However, the term oligonucleotide or polynucleotide additionally includes analogous structures, such as, for example, peptide nucleic acids. Accordingly, there may be used in the conjugate according to the invention also any possible oligonucleotide-analogous structures that have at least one chemical modification as compared with naturally occurring molecules. The expression “chemical modification” means that the conjugate-containing nucleic acid species according to the invention has been modified in comparison with naturally occurring nucleic acid species by the replacement, insertion (addition) or removal of individual or a plurality of atoms.

The form of the chemical modification is preferably such that the nucleic acid contains at least one analogue of naturally occurring nucleotides.

In a list which is in no way conclusive, the following may be mentioned as examples of nucleotide analogues which can be used according to the invention: phosphoramidates, phosphorthioates, peptide nucleotides, methyl phosphonates, 7-deazaguanosine, 5-methylcytosine and inosine as well as 2-alkoxy-substituted ribonucleotide species.

In the case of the conjugate according to the invention comprising the dithiolane derivative and the above nucleoside/nucleotide species (that is to say mononucleosides, mononucleotides, oligonucleotides and polynucleotides), the dithiolane derivative, especially a lipoic acid derivative, can be conjugated via the 5-carbon atom of the corresponding nucleoside/nucleotide or via the terminal 5-carbon atom of the oligo- or poly-nucleotide (the nucleoside, nucleotide, oligo- or poly-nucleotide replacing the group —OR⁵ in formula A above) or it can be conjugated via the 3-carbon atom of the nucleoside/nucleotide or via the terminal 3-carbon atom of the oligo- or poly-nucleotide (the nucleoside, nucleotide, oligo- or poly-nucleotide replacing the group —OR⁴ in formula A above).

For the 3-terminal dithiolane conjugation of nucleic acid species there is suitable especially a dithiolane derivative, such as a corresponding lipoic acid derivative, that is bonded via a corresponding coupling group according to the invention to a suitable polymer carrier. A particularly preferred example of a dithiolane derivative that is particularly suitable for 3-terminal conjugation is shown in formula I above.

For the conjugation according to the invention of a dithiolane derivative having the above definition with biomolecules, especially oligonucleotides and polynucleotides, there is suitable especially a dithiolane-substituted, preferably lipoic acid-substituted, phosphorous acid ester amide reagent, which allows the dithiolane moiety, for example a lipoic acid moiety, to be attached terminally and/or internally at any desired position of oligonucleotide chains. Accordingly, with the aid of the present invention, corresponding molecules, especially oligonucleotides, can be dithiolanylated, especially lipoylated, one or more times. A specific example of a dithiolane derivative according to the invention that is particularly suitable for this purpose is shown in formula II above.

The conjugates, e.g. lipoylated oligonucleotides, according to the invention are advantageous over known thiol conjugates in that, for example, they can readily be stored over a prolonged period, while the conventional thiol conjugates are stable only under inert conditions.

The present invention further provides a process for the preparation of the dithiolane derivative defined above, comprising the steps:
(a) preparation of a dithiolane compound of formula B 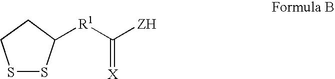
   wherein R¹ and X are as defined in formula A above and Z=NH or O,
(b) activation of the carbonyl or sulfonyl group of the dithiolane compound and
(c) reaction of the activated dithiolane compound with a compound of formula C 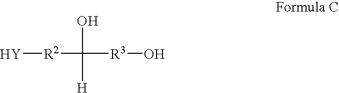
   wherein R², R³ and Y are as defined in formula A above.

The further introduction of the moieties R⁴ and R⁵ into the resulting dithiolane diol compound is carried out by processes known to a person skilled in the art.

According to a preferred embodiment of the process according to the invention, activation of the carbonyl or sulfonyl group of the dithiolane compound according to formula B above is carried out by converting it into an activated ester, especially into a dicarboxylic acid imide ester, such as a NHS ester. By activating the compound according to formula B, for example lipoic acid, by means of a suitable dicarboxylic acid imide, such as hydroxysuccinimide, to form the corresponding ester, a considerable increase in the yield in the synthesis of the central compound according to formula C above is achieved. Alternatively, activation of the dithiolane compound according to formula B above may also be effected, for example, using a carbodiimide compound, such as dicyclohexylcarbodiimide (DCC). According to a further embodiment of the preparation process according to the invention, the dithiolane compound according to formula B above can be converted into a corresponding acid halide, for example by reacting the dithiolanoic acid compound with oxalyl chloride to form the corresponding acid chloride. Because the resulting acid chloride is frequently not very stable, the above steps (b) and (c) are advantageously carried out simultaneously by adding to the compound of formula B the corresponding agent for preparing the acid halide and the corresponding diol spacer compound.

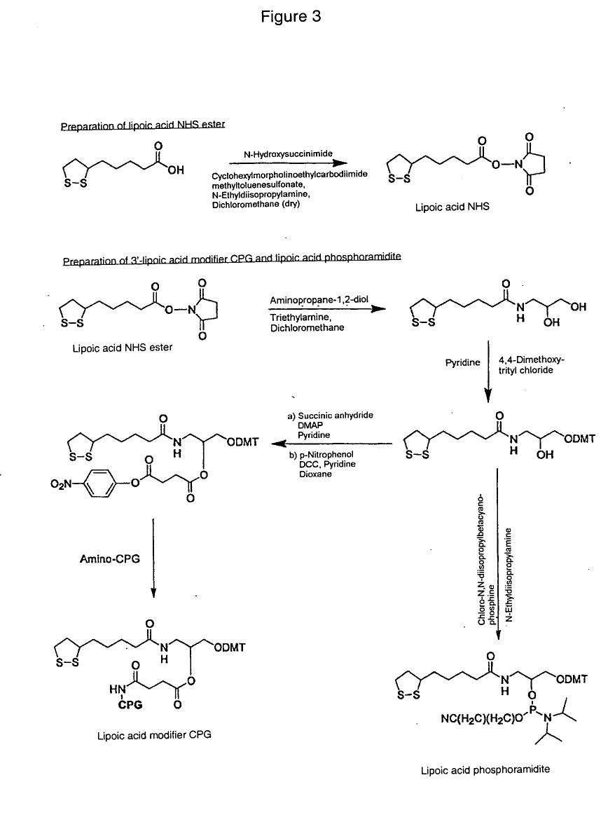

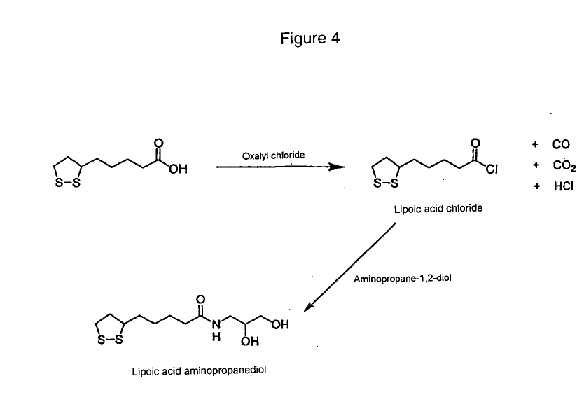

The preparation of the dithiolane derivative according to the invention by means of activation of the dithiolane starting compound by a carbodiimide is shown hereinbelow using the example of the preparation of the dithiolane derivative of formula I above from lipoic acid, aminopropane-1,2-diol and amino-CPG (Scheme 1 according to FIG. 1). In a further scheme, the corresponding preparation of the above phosphoramidite derivative according to formula II is shown (Scheme 2 according to FIG. 2). Scheme 3 hereinbelow (FIG. 3) shows by way of example the process according to the invention for the preparation of the dithiolane derivative with the aid of the conversion of lipoic acid to a NHS ester, the further synthesis steps to form a 3-lipoic acid modifier CPG or a lipoic acid phosphoramidite also being shown. The preparation of lipoic acid aminopropanediol is shown in Scheme 4 (FIG. 4) as an example of the preparation of the dithiolane derivative according to the invention from dithiolanoic acid, in this case lipoic acid, by means of oxalyl chloride and reaction with aminopropane-1,2-diol.

The present invention also includes a process for the preparation of the conjugate according to the invention, which comprises the steps:
(i) preparation of the dithiolane derivative according to the invention and
(ii) reaction of the dithiolane derivative with an organochemical or biochemical molecule containing at least one amino or hydroxyl group.

The preparation of the dithiolane derivative is preferably carried out by the preparation process defined above. The organochemical or biochemical compound containing at least one amino or hydroxyl group is preferably one of the compounds mentioned hereinbefore.

The process according to the invention for the preparation of the conjugate is particularly preferred when there is used a dithiolane derivative containing a dicarbonyl group which bonds the dithiolane-bridging member-spacer construct to a polymer carrier via an amide or ester bond. A specific example of such a compound is shown in formula I above.

According to this particularly preferred embodiment of the present invention, the lipoic acid moiety is bonded via an amide bond to the amino group of a 3-aminopropane-1,2-d ol spacer. One of the hydroxyl groups of the spacer is substituted by a protecting group, here dimethoxytrityl, which is selectively cleaved off before the synthesis of an oligonucleotide chain. The other hydroxyl group of the 3-aminopropane-1,2-diol spacer is bonded to a group known from the prior art which is used for anchoring oligonucleotides to polymer carriers containing amino or hydroxyl groups, for example a succinyl moiety.

After cleaving off of the 4,4-dimethoxytrityl group by a process known in the prior art, synthesis of the oligonucleotide chain is carried out, starting from the freed hydroxyl group, according to the prior art, it being unimportant whether the synthesis takes place from the 3′ end or the 5′ end. After the synthesis of a corresponding nucleic acid chain, for example an oligonucleotide chain, with cleaving off, known in the prior art, from the polymer carrier, for example using ammonia or amines, the moiety containing the lipoic acid or dithiolane group remains, according to the invention, bonded to the oligonucleotide chain via the spacer.

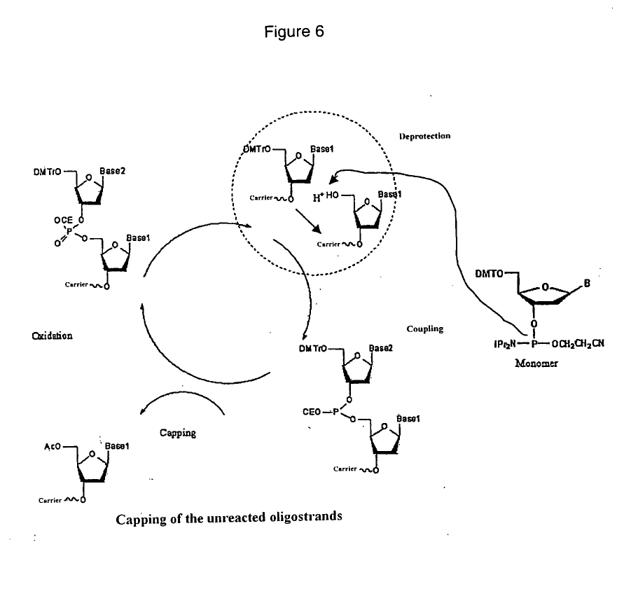

A further preferred process for the preparation of a conjugate according to the invention uses the dithiolane derivative in which R⁴ is H or an acid-labile protecting group, for example a dimethoxytrityl group, and R⁵ is a phosphoramidite group, especially N,N-diisopropyl-beta-cyanoethoxy-phosphoramidite. A specific embodiment of a dithiolane derivative according to the invention, in this case a lipoic acid derivative, is shown in formula II above. Here too, one hydroxyl group of the spacer is protected with a corresponding protecting group, for example dimethoxytrityl. The second hydroxyl group is bonded to an activatable moiety known from the prior art, for example a phosphoramidite moiety, in formula II a (beta-cyanoethoxy)diisopropylamino-phosphane moiety. According to the invention, this activatable moiety permits linking to a hydroxyl or amino group. Following this reaction step, the dimethoxytrityl protecting group is cleaved off according to known processes. The hydroxyl function so obtained can then optionally be used for the linking of a further nucleotide unit. The dithiolane derivatives according to the invention are therefore very particularly suitable for the preparation of corresponding nucleic acid conjugates, with oligonucleotides being particularly preferred. The dithiolane derivative of the present invention, comprising the specific bridging member-diol-spacer structure, has the advantage over the reagents known in the prior art that it can be used in a simple manner in standard nucleic acid synthesis processes, such as, for example, in the phosphoramidite method and anchoring to polymer carriers, etc. Such processes are described, for example, by Gait (Oligonucleotide synthesis: a practical approach, IRL Press, Oxford, 1987), and the relevant content of the disclosure of this specification is incorporated in its entirety into the present invention. The course of a synthesis cycle in the phosphoramidite method is shown by way of example in FIG. 6.

The dithiolane derivative according to the invention is suitable especially for immobilising organochemical or biochemical molecules on noble metals or noble metal alloys and also on semiconductors.

Accordingly there is also provided according to the invention a coated noble metal structure which comprises
(a) a substrate of a carrier material,
(b) at least one layer, formed at least on a partial area of the substrate, of one or more noble metals and/or noble metal alloys, and
(c) at least one dithiolane derivative according to the invention immobilised at least on a partial area of the noble metal layer and/or at least one conjugate according to the invention having the above definition, immobilised at least on a partial area of the noble metal layer.

There is further provided according to the invention a coated semiconductor structure, comprising a thiol-binding semiconductor carrier and at least one dithiolane derivative according to the invention immobilised at least on a partia area of the semiconductor and/or at least one conjugate according to the invention having the above definition, immobilised at least on a partial area of the semiconductor. Preferred semiconductor structures according to the invention are those in which gallium arsenide or indium phosphide is coated with dithiolane derivatives or conjugates according to the invention; with regard to the binding of thiols on semiconductors see e.g. Lunt et al. (1991) J. Appl. Phys. 70: 7449; Gu et al. (1995) Langmuir 11: 1849-1851; Zerulla et al. (1999) Langmuir 15, 5285-5294.

Preference is given according to the invention to those layer structures in which the immobilised dithiolane derivative and/or the immobilised conjugate form(s) a self-assembled monolayer (SAM). This embodiment of the coated noble metal or semiconductor structure according to the invention provides a controlled layer of the immobilised molecules, for example biomolecules such as oligonucleotides. By the formation of a SAM it is possible to control the density of the immobilised molecules in such a manner as to achieve as balanced a relationship as possible between a high density of, for example, probe molecules used in an array (i.e. as large a number as possible of binding sites for potential binding partners (target molecules) per unit area) and their accessibility to the target molecules to be investigated, which gives a high signal intensity when used in a biochip (e.g. a corresponding microarray).

In order in particular to increase the hybridisation efficiency in the case of immobilised probe oligonucleotides, the layer structure can be so formed according to the invention that, in addition to the dithiolane derivative or conjugate according to the invention, for example a spacer (see Southern et al. (1999) The Chiping Forecast 21: 5-9; Southern et al. (1997) Nucl. Acid Res. 25: 1155-1161) is added. The hybridisation efficiency can further be increased, for example, by the use of probe oligos loosely packed at the surface (Southern et al. (1997), supra).

In comparison with other immobilisation strategies, it is possible in the case of the oligonucleotides conjugated according to the invention to form a so-called mixed oligo-SAM by adding, in addition to the dithiolane derivative according to the invention or the conjugate thereof with an oligonucleotide, a conventional alkanethiol for controlling the density of the desired oligo-SAM. As a result, the oligonucleotide used is likewise readily accessible for efficient hybridisation. By means of these measures, the hybridisation efficiency can be increased to up to 100% (Levicky (1998) JACS. 120: 9787-9792).

A further major advantage of the dithiolane-modified molecules, especially of lipoic acid-modified oligonucleotides, immobilised on a noble metal is the high binding capacity on the noble metal. For example, gold has a lipoic acid binding capacity of 7.1×10¹⁰ mol/cm² (Pirrung (2002) Angew. Chem. 114: 1326-1341). The layer structures according to the invention are also superior to those of the prior art, especially conventional thiol-modified oligonucleotide layers, in that, owing to the presence of the vicinal dithiol group, the dithiolane derivative of the present invention exhibits a higher bond strength on the carrier material, and a more stable bond is therefore to be expected owing to the double anchoring (see Wang et al. (1998), supra; Cheng et al. (1995), supra).

The coated noble metal or semiconductor structures according to the invention are further distinguished by the fact that the constituents and also the structure itself can be prepared by standard synthesis processes. By contrast, conventional thiol-modified oligonucleotides from the prior art require complex treatment or working up in order that the —SH group desired for chemisorption of the corresponding conjugate is freed.

For the coated noble metal structure according to the invention there are suitable a number of noble metals, such as gold, silver, copper, platinum, palladium, ruthenium and iridium, as well as alloys of noble metals. A particularly preferred noble metal according to the invention is, for example, gold. The coated noble metal structure according to the invention may be in such a form that at least a partial area of the substrate is flat. As carrier materials there are used especially glass, plastics, semiconductors (especially silicon) and metals, preferably metals different from the noble metal of the coating. If required, the surface of the carrier material may be treated or coated according to a process known to a person skilled in the art, in order to bind the noble metal or noble metal alloy to be applied. At present, glass carriers are used as standard for biochips, especially microarrays. On the other hand, particles of noble metals or noble metal alloys or particulate carrier materials coated therewith are also suitable, so that the substrate according to the invention may also be in particle form. When the coated noble metal structure according to the invention is in particle form, materials such as, for example, polymers, e.g. polystyrene, glass, silica etc. are particularly suitable.

In the case of flat structures, the noble metal or semiconductor structure is particularly suitable for use as a nucleic acid array, especially as a DNA microarray, on biochips.

Accordingly, there is also provided according to the invention a biochip which comprises the coated noble metal/semiconductor structure according to the invention.

Further advantages of the coating according to the invention of noble metals, especially gold, with dithiolane derivatives and conjugates derived therefrom, especially oligonucleotides, are a particularly good binding capacity (Wolfl (2000) transcript Laborwert 3), the possibility of forming SAMs (Bamdad (1998) Biophys. J. 75: 1997-2003), i.e. the controlled binding of analytical molecules, especially DNA, for use in diagnostics, as well as the possibility of rapidly producing a biochip, because the immobilisation of the desired analytical molecules, such as, for example, DNA oligonucleotides, and blocking of the chip surface is possible within about 3 hours.

The figures show:

FIG. 1 shows, in a synthesis scheme, the preparation of a dithiolane derivative according to the invention, here a CPG modifier reagent according to formula I (Scheme 1). Lipoic acid is reacted with aminopropane-1,2-diol with activation by DCC, the corresponding propane-2,3-diol-1,2-dithiolane-3-pentanoic acid amide being obtained. In a further reaction, one of the hydroxyl groups is protected with 4,4-dimethoxytrityl chloride. A succinyl moiety is attached to the hydroxyl group that has remained free by reaction with succinic anhydride, and a p-nitrophenyl ester is then prepared by activation with DCC. Finally, the ester so activated is reacted with amino-CPG to give the dithiolane derivative of formula I coupled to the polymer carrier.

FIG. 2 shows, in a synthesis scheme 2, the preparation of a dithiolane derivative according to the invention that has a phosphoramidite group on a hydroxyloxygen of the diol spacer, the starting material used being the DMT-protected propane-2,3-diol-1,2-dithiolane-3-pentanoic acid amide, which can be prepared according to Scheme 1 of FIG. 1. The lipoic acid amide-spacer conjugate is reacted with chloro-N,N-diisopropyl-beta-cyanophosphine and N,N-diisopropylamine in dry dichloromethane in order to give the compound of formula II.

FIG. 3 shows, in a further synthesis scheme, the coupling of lipoic acid with aminopropane-1,2-diol in an alternative method of preparing 3-lipoic acid modifier CPG or lipoic acid phosphoramidite (Scheme 3). First of all, lipoic acid is activated by means of hydroxysuccinimide to form lipoic acid NHS ester. The NHS ester is then reacted with aminopropane-1,2-diol in dichloromethane/triethylamine to give the lipoic acid amide-spacer derivative again. A 4,4-dimethoxytrityl protecting group is then attached to the terminal hydroxyl moiety. Starting from this protected derivative it is then possible to prepare, for example, the corresponding 3-lipoic acid modifier CPG or a lipoic acid phosphoramidite, as shown in FIG. 1 (Scheme 1) or FIG. 2 (Scheme 2).

FIG. 4 shows, in a further synthesis scheme, a different embodiment of the preparation of lipoic acid aminopropanethiol (Scheme 4). In this case, lipoic acid is reacted with oxalyl chloride to give the corresponding acid chloride, in order thus to obtain an activated compound which reacts with aminopropane-1,2-diol to give the desired product.

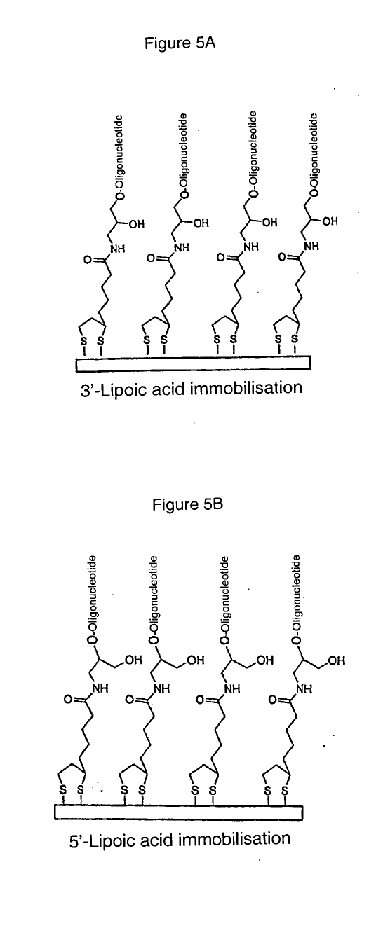

FIG. 5A shows, in a diagrammatic representation, the immobilisation of oligonucleotide-lipoic acid conjugates according to the invention, the lipoic acid derivative being bonded to the 3-carbon atom of the terminal nucleotide.

FIG. 5B shows, in a representation corresponding to FIG. 5A, the conjugation of the lipoic acid derivative at the terminal 5-carbon atom of the conjugated oligonucleotide.

FIG. 6 shows, in a synthesis scheme, the oligonucleotide synthesis cycle carried out in the phosphoramidite method (see Gait (1987), supra).

The implementation examples which follow explain the present invention further, without limiting it.

### IMPLEMENTATION EXAMPLE 1

Preparation of 3′-Lipoic Acid Modifier CPG

Literature:

Nelson et al. (1989) Nucl. Acids Res. 17: 7179-7186; Nelson et al. (1989) Nucl. Acids Res. 17: 7187-7194

Chemicals Used:

D,L-alpha-lipoic acid, 4-pyrrolidinopyridine, 3-aminopropane-1,2-diol, dicyclohexylcarbodiimide, dichloromethane, 4,4-dimethoxytriphenylmethyl chloride, pyridine (dry), methanol, dimethylaminopyridine, succinic anhydride, ethyl acetate, doxane, p-nitrophenol, triethylamine, aminopropyl-CPG (preparation see under Amino-CPG, http://www.interactiva.de), dimethylformamide, acetic anhydride, diethyl ether.

Preparation of propane-2,3-diol-1,2-dithiolane-3-pentanoic acid amide

D,L-alpha-lipoic acid (5 mmol, 1 g) was added to a mixture consisting of 3-aminopropane-1,2-diol (5 mmol, 0.5 g), 4-pyrrolidinopyridine (5 mmol, 0.7 g) and dicyclohexylcarbodiimide (10 mmol, 1.9 g) in 20 ml of dichloromethane, and stirring was carried out overnight at room temperature. The resulting dicyclohexylurea was filtered off and the solvent was evaporated off using a rotary evaporator.

Preparation of propane-2,3-diol-3-O-4,4-dimethoxydiphenylmethyl-1,2-dithiolane-3-pentanoic acid amide

The viscous, impure product was dissolved in 10 ml of dry pyridine and reacted with 4,4-dimethoxytriphenylmethyl chloride (5 mmol, 1.69 g), and stirring was carried out overnight at room temperature.

10 ml of methanol were then added and the mixture was stirred for a further 10 minutes and dried in vacuo.

The product was purified by means of a silica gel column (5×20 cm) (yield 61%, elution mixture: dichloromethane/hexane/triethylamine (50/50/1%)) and examined by means of thin layer chromatography (TLC) (eluant: dichloromethane/hexane/triethylamine, 50/50/1%), detection: UV light (the trityl group can be made visible under HCl vapour).

Preparation of 3-Lipoic Acid Modifier CPG

Succinic anhydride (3 mmol, 300 mg) was added to a solution of propane-2,3-diol-3-O-4,4-dimethoxytriphenylmethyl-1,2-dithiolane-3-pentanoic acid amide (1 mmol, 0.61 g) and dimethylaminopyridine (0.9 mmol, 200 mg) in dry pyridine (12 ml), and stirring was carried out overnight at room temperature. The mixture was dissolved in ethyl acetate (100 ml), washed with a 0.5 M sodium chloride solution (3×100 ml) and a saturated sodium chloride solution (1×100 ml) and dried over sodium sulfate. The solvent was removed and the product was dried in vacuo.

The dry succinic acid derivative, p-nitrophenol (2.5 mmol, 350 mg) and 0.5 ml of dry pyridine were dissolved in 10 ml of dry dioxane. Dicyclohexylcarbodiimide (4.8 mmol, 1.0 g) was then added to the mixture and stirring was carried out for 3 hours under mild conditions. The dicyclohexylurea so formed was filtered off.

1 g of aminopropane-CPG was suspended in the filtrate, 1 ml of triethylamine was added, and shaking was carried out overnight at room temperature.

The derivatised carrier was washed carefully with dimethylformamide, methano and diethyl ether and dried.

Finally, the lipoic acid carrier was capped with a solution of acetic anhydride/-pyridine/dimethylaminopyridine (10:90:1). Thorough washing was then carried out with methanol and diethyl ether, followed by thorough drying under a high vacuum.

The synthesis is shown in Scheme 1 (FIG. 1).

### IMPLEMENTATION EXAMPLE 2

Preparation of Lipoic Acid Phosphoramidite

Literature:

Nelson et al., (1989) Nucl. Acids Res. 17: 7179-7186

Chemicals Used:

N,N-diisopropylethylamine, dichlormethane (dry), chloro-N,N-diisopropyl-beta-cyanoethoxyphosphoramidite, methanol, ethyl acetate, hexane, triethylamine

The starting material to be used is propane-2,3-diol-O-4,4-dimethoxytriphenyl-methyl-1,2-dithiolane-3-pentanoic acid amide, which is prepared, for example, according to the first implementation example.

Preparation of propane-2,3-diol-2-O-[N,N-diisopropyl-beta-cyanoethoxyphosphoramidite]-3-O-[4,4-dimethoxyydiphenylmethyl]-1,2-dithiolane-3-pentanoic acid amide

Propane-2,3-diol-3-O-4,4-dimethoxytriphenylmethyl-1,2-dithiolane-3-pentanoic acid amide (1.25 mmol, 2.2 μg), dried in vacuo, and dry N,N-diisopropylethyl-amine (6 mmol, 0.78 g) were dissolved, under argon, in 45 ml of dry dichloromethane. Chloro-N,N-diisopropyl-beta-cyanoethoxyphosphoramidite (2.5 mmol, 0.6 g) was slowly added to this solution in the course of 5 minutes, and stirring was carried out for one hour under argon. Methanol (1 ml) was then added, and stirring was continued for a further 10 minutes. The reaction mixture was partitioned between ice-cold ethyl acetate (170 ml) and 10% ice-cold sodium hydrogen carbonate (275 ml). The organic phase was washed with 10% cold sodium hydrogen carbonate solution (275 ml) and dried over sodium sulfate. After removal of the solvent in vacuo, the rubber-like residue was purified by means of a silica gel column (5×10 cm). The desired product could be eluted isocratically with dichloromethane/hexane/triethylamine (50:50:1%) (examination by means of TLC, eluant: dichloromethane/hexane/triethylamine (50:50:1%), detection: UV light, it being possible to make the trityl group visible under HCl vapour). The solvent was concentrated using a rotary evaporator and the purified lipoic acid phosphoramidite was dried in vacuo. The synthesis is shown in Scheme 2 (FIG. 2).

### IMPLEMENTATION EXAMPLE 3

Synthesis of 3-, 5′-Lipoylated Oligonucleotides

The oligonucleotide syntheses were carried out according to a standard protocol on an Expedite TM synthesizer (PerSeptive Biosystems). All the synthesised oligonucleotides were subsequently purified in the standard manner by means of HPLC. The syntheses of 3-, 5-lipoylated oligonucleotides could be confirmed by MALDI.

Implementation Example 4:

Immobilisation of Lipoylated Oligonucleotides on Gold-Coated Carriers

Gold Carriers

Commercially available microscope slides which had been coated by sputtering with 10 nm of tungsten/titanium (adhesion layer) and 100 nm of gold were used as the substrate. The gold carriers were cleaned with Piranha solution (70% H₂SO₄: 30% H₂O₂).

Immobilisation

1, 2, 5, 10, 15 and 30 pmol of oligonucleotides modified in 3 μl of 1 M NaH₂PO₄ (pH 4.1) with lipoic acid at 3-C and 5-C (Lip-oligonucleotides) were applied to cleaned gold carriers. The gold carriers were left in a chamber saturated with steam for 2 hours. After immbobilisation, the drops of liquid were removed using a pipette and thorough rinsing was carried out using bidistilled water.

Passivation of the Carrier Periphery

After immobilisation with Lip-oligonucleotides, the gold carrier was incubated for 45 minutes in a 1 mM solution of thio-polyethylene glycol MW 5000 (Rapp Polymers) in order to prevent non-specific binding. It was then rinsed carefully with bidistilled water. Any remaining drops of water were blown off with nitrogen.

Demonstration of Immobilisation

In order to demonstrate the immobilisation, the coated microscope slide was hybridised with radioactively labelled oligonucleotides containing the complementary sequence to the immobilised nucleotides (hybridisation with ³²P, 1 M NaCl-TE buffer, overnight at room temperature). Immobilisation of lipoylated oligonucleotides could additionally be confirmed with the aid of X-ray photoelectron spectroscopy (XPS) examinations.

1. Dithiolane derivative of formula A 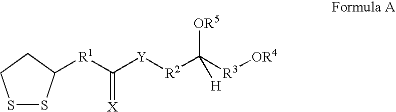
wherein
R¹ is not present or is a straight-chain or branched, saturated or unsaturated, substituted or unsubstituted aliphatic, heteroaliphatic moiety having from 1 to 20 carbon atoms or a corresponding substituted or unsubstituted aromatic moiety,
R², R³ are each independently of the other a straight-chain or branched, saturated or unsaturated, substituted or unsubstituted aliphatic or heteroaliphatic moiety having from 1 to 10 carbon atoms or a corresponding substituted or unsubstituted aromatic moiety,
R⁴, R⁵ are each selected independently of one another from the group consisting of H, acid-labile protecting groups, and amino and/or hydroxy coupling groups,
X is O or S and
Y is NH or O.

2. Dithiolane derivative according to claim 1, wherein R¹ is tetramethylene.

3. Dithiolane derivative according to claims 1 or 2, wherein X=O and Y═N.

4. Dithiolane derivative according to claim 1, wherein R² is (CH₂)ₙ, wherein n is an integer from 1 to 10, preferably from 1 to 5.

5. Dithiolane derivative according to claim 4, wherein R² is methylene.

6. Dithiolane derivative according to claim 1, wherein R³ is (CH₂)ₘ, wherein m is an integer from 1 to 10, preferably from 1 to 5.

7. Dithiolane derivative according to claim 6, wherein R³ is methylene.

8. Dithiolane derivative according to claim 1, wherein R⁴ is H and R⁵ is an acid-labile protecting group, or vice versa.

9. Dithiolane derivative according to claim 1, wherein R⁴ is H or an acid-labile protecting group and R⁵ is a dicarbonyl group, or vice versa.

10. Dithiolane derivative according to claim 9, wherein the dicarbonyl group is bonded to a polymer carrier via an amide or ester bond.

11. Dithiolane derivative according to claim 10 having the following formula: 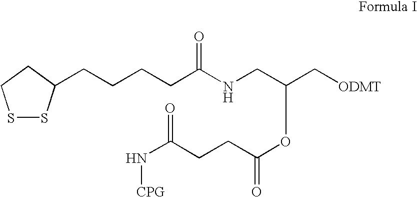
wherein DMT is a dimethoxytrityl group.

12. Dithiolane derivative according to claims 1, wherein R⁴ is H or an acid-labile protecting group and R⁵ is a phosphoramidite group, or vice versa.

13. Dithiolane derivative according to claim 12, wherein the phosphoramidite group is N,N-diisopropyl-β-cyanoethoxyphosphor-amidite.

14. Dithiolane derivative according to claim 13 having the following formula: 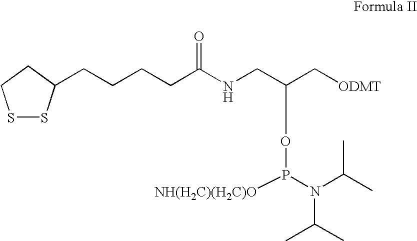
wherein DMT is a dimethoxytrityl group.

15. Conjugate of at least one dithiolane derivative according to claim 1 and an organochemical or biochemical molecule containing at least one amino or hydroxyl group, which molecule replaces the group —OR⁴ or the group —OR⁵ in formula A via the amino or hydroxyl group.

16. Conjugate according to claim 15, wherein the biochemical molecule is selected from the group consisting of nucleosides, nucleotides, oligonucleotides and polynucleotides.

17. Conjugate according to claim 16, wherein the dithiolane derivative is conjugated via the 5-carbon atom of the nucleoside/nucleotide or via the terminal 5-carbon atom of the oligo- or polynucleotide and replaces the group —OR⁵ in formula A.

18. Conjugate according to claim 16, wherein the dithiolane derivative is conjugated via the 3-carbon atom of the nucleoside/nucleotide or via the terminal 3-carbon atom of the oligo- or polynucleotide and replaces the group —OR⁴ in formula A.

19. Process for the preparation of a dithiolane derivative according to claim 1, comprising the steps:
(a) preparation of a dithiolane compound of formula B 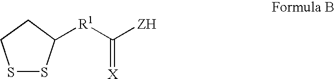
wherein R¹ and X are as defined in formula A and Z=NH or O,
(b) activation of the carbonyl or sulfonyl group of the dithiolane compound and
(c) reaction of the activated dithiolane compound with a compound of formula C 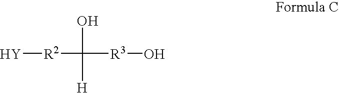
wherein R², R³ and Y are as defined in formula A.

20. Process according to claim 19, wherein in step (b) the compound of formula B is converted into an activated ester.

21. Process according to claim 20, wherein, the activated ester is a dicarboxylic acid imide ester.

22. Process according to claim 19, wherein in step (b) the compound of formula B is converted into the acid halide.

23. Process according to claim 22, wherein steps (b) and (c) are carried out simultaneously.

24. Process for the preparation of the conjugate according to claim 15, comprising the steps:
(i) preparation of the dithiolane derivative and
(ii) reaction of the dithiolane derivative with an organochemical or biochemical molecule containing at least one amino or hydroxyl group.

25. Process according to claim 24, wherein the preparation of the dithiolane derivative is carried out by a process comprising the steps of:
(a) preparation of a dithiolane compound of formula B 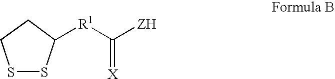
wherein R¹ and X are as defined in formula A and Z=NH or O,
(b) activation of the carbonyl or sulfonyl group of the dithiolane compound and
(c) reaction of the activated dithiolane compound with a compound of formula C 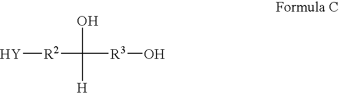
wherein R², R³ and Y are as defined in formula A.

26. Process according to claim 24, wherein R⁴ of the dithiolane derivative is H or an acid labile protecting group and R⁵ is a dicarbonyl group, or vice versa, and wherein the dicarbonyl group is bonded to a polymer carrier via an amide or ester bond.

27. Process according to claim 24, wherein R⁴ of the dithiolane derivative is H or an acid labile protecting group and R⁵ is a phosphoramidite group, or vice versa.

28. Process according to of claim 24, wherein the biochemical molecule is selected from the group consisting of nucleosides, nucleotides, oligonucleotides and polynucleotides.

29. Coated noble metal structure comprising
(A) a substrate of a carrier material,
(B) at least one layer, formed at least on a partial area of the substrate, of one or more noble metals and/or noble metal alloys and
(C) at least one dithiolane derivative according to claim 1 immobilised at least on a partial area of the noble metal layer, and/or at least one conjugate of said dithiolane derivative and an organochemical or biochemical molecule containing at least one amino or hydroxyl group, which molecule replaces the group —OR⁴ or the group —OR⁵ in formula A via the amino or hydroxyl group immobilised at least on a partial area of the noble metal layer.

30. Noble metal structure according to claim 29, wherein the immobilised dithiolane derivative and/or the immobilised conjugate form(s) a self-assembled monolayer.

31. Noble metal structure according to claim 29, wherein the noble metal is selected from the group consisting of gold, silver, copper, platinum, palladium, ruthenium and iridium, and alloys of these noble metals.

32. Noble metal structure according to claim 31, wherein the noble metal is gold.

33. Noble metal structure according to claim 29,
wherein at least a partial area of the substrate is flat.

34. Noble metal structure according to claim 29,
wherein the substrate is in particle form.

35. Noble metal structure according to claim 29,
wherein the carrier material is glass, plastics, a semiconductor or a metal.

36. Coated semiconductor structure, comprising a thiol-binding semiconductor carrier and at least one dithiolane derivative according to claim 1 immobilised at least on a partial area of the semiconductor carrier and/or at least one conjugate of said dithiolane derivative and an organochemical or biochemical molecule containing at least one amino or hydroxyl group which molecule replaces the group —OR⁴ or the group —OR⁵ in formula A via the amino or hydroxyl group immobilised at least on a partial area of the semiconductor carrier.

37. Semiconductor structure according to claim 36, wherein the semiconductor is gallium arsenide or indium phosphide.

38. Semiconductor structure according to claim 36, wherein the immobilised dithiolane derivative and/or the immobilised conjugate form(s) a self-assembled monolayer.

39. Biochip, comprising the noble metal structure according to claim 29 and/or the semiconductor structure according to claim 36.

40. (canceled)
